(19) United States
(12) Patent Application Publication
(10) Pub. No.: US 2026/0237066 A1
(43) Pub. Date: Aug. 13, 2026

(54) ATTENUATION CORRECTION FOR MEDICAL IMAGING

(71) Applicant: Siemens Medical Solutions USA, Inc., Malvern, PA (US)

(72) Inventors: Joshua Schaefferkoetter, Knoxville, TN (US); Michael E. Casey, Louisville, TN (US); Charles Hayden, Knoxville, TN (US)

(21) Appl. No.: 19/160,683

(22) PCT Filed: Jun. 9, 2023
(86) PCT No.: PCT/US2023/068163

§ 371 Date: Aug. 28, 2025

(51) Int. Cl. G06T 7/00

(52) U.S. Cl. CPC G06T 7/0012; G06T 2207/10081; G06T 2207/10088; G06T 2207/10108; G06T 2207/20081; G06T 2207/20084

(57) ABSTRACT

A framework for attenuation correction. An attenuation map may be generated by applying a non-attenuation corrected emission image to one or more trained artificial neural networks. Attenuation correction may be performed on the non-attenuation corrected emission image by using the attenuation map.

### TECHNICAL FIELD

The present disclosure generally relates to medical image data processing, and more particularly to a framework for attenuation correction.

### BACKGROUND

The field of medical imaging has seen significant advances since the time X-Rays were first used to determine anatomical abnormalities. Medical imaging hardware has progressed in the form of newer machines such as Medical Resonance Imaging (MRI) scanners, Computed Axial Tomography (CAT) scanners, etc. Digital medical images are constructed using raw image data obtained from such scanners. Digital medical images are typically either a two-dimensional (“2-D”) image made of pixel elements or a three-dimensional (“3-D”) image made of volume elements (“voxels”). Because of large amounts of image data generated in any given scan, there has been and remains a need for developing image processing techniques that can automate some or all of the processes to determine the presence of anatomical abnormalities in scanned medical images.

Multimodality imaging plays an important role in accurately identifying diseased and normal tissues. Multimodality imaging provides combined benefits by fusing images acquired by different modalities. The complementarity between anatomic (e.g., computed tomography (CT), magnetic resonance (MR)) and molecular (e.g., positron-emission tomography (PET), single-photon emission computerized tomography (SPECT)) imaging modalities, for instance, has led to the widespread use of PET/CT and SPECT/CT imaging.

Serial PET/CT (or SPECT/CT) involves a sequence of multiple scans (or multi-scans) to assess the status of the region of interest over a period of time. For cardiac PET/CT, two scans are usually performed in one imaging session. The first scan is typically a “rest” scan that is performed when the patient is at rest; the second scan is typically a “stress” scan performed when the patient has been given a pharmacological stress agent. The usual workflow involves first acquiring a low dose CT for attenuation correction. Subsequently, the patient is administered with the radiotracer (e.g., 82Rb, 13NH3) and list-mode PET data is simultaneously acquired. After a short wait for the radiotracer to clear, the patient is given the stress agent. Once the stress agent has increased the heartrate, a second injection is performed along with the acquisition of the “stress” list-mode PET data. In many sites, a second CT scan is performed after the “stress” scan to provide more accurate attenuation correction for the “stress” images.

A mismatch between the CT and PET image data can occur due to breathing motion and/or voluntary patient motion. In particular, the stress agent can cause the heart to move and generate some patient discomfort. Both of these effects can cause mismatch between the PET and CT image data. An additional problem can occur due to the CT field of view (FOV) being limited by the size of the CT detector. Even though there is an algorithm to extend the CT image beyond the detector FOV, artifacts can occur with large patients that compromise attenuation correction.

Image reconstruction is typically performed without attenuation correction. A registration program may move the CT image in x, y and/or z dimensions to produce the best match between the non-attenuation corrected (NAC) images and the CT images. NAC and CT images are displayed as overlayed images and the operator decides if the registration is satisfactory. If not, the program may allow the operator to manually move the CT image to the proper position. Since more movement is expected due to the stress agent, some centers opt for a second CT scan after the stress scan. The same workflow typically applies with an automated registration followed by manual intervention.

### SUMMARY

Described herein is a framework for attenuation correction. An attenuation map may be generated by applying a non-attenuation corrected emission image to one or more trained artificial neural networks. Attenuation correction may be performed on the non-attenuation corrected emission image by using the attenuation map.

### BRIEF DESCRIPTION OF THE DRAWINGS

A more complete appreciation of the present disclosure and many of the attendant aspects thereof will be readily obtained as the same becomes better understood by reference to the following detailed description when considered in connection with the accompanying drawings.

FIG. 1 shows a block diagram illustrating an exemplary system;

FIG. 2 shows an exemplary method of attenuation correction;

FIG. 3 illustrates an exemplary application of the present framework for a cardiac scan; and

FIG. 4 shows an exemplary application of the present framework for a series of physiologically gated images.

### DETAILED DESCRIPTION

In the following description, numerous specific details are set forth such as examples of specific components, devices, methods, etc., in order to provide a thorough understanding of implementations of the present framework. It will be apparent, however, to one skilled in the art that these specific details need not be employed to practice implementations of the present framework. In other instances, well-known materials or methods have not been described in detail in order to avoid unnecessarily obscuring implementations of the present framework. While the present framework is susceptible to various modifications and alternative forms, specific embodiments thereof are shown by way of example in the drawings and will herein be described in detail. It should be understood, however, that there is no intent to limit the invention to the particular forms disclosed, but on the contrary, the intention is to cover all modifications, equivalents, and alternatives falling within the spirit and scope of the invention. Furthermore, for ease of understanding, certain method steps are delineated as separate steps; however, these separately delineated steps should not be construed as necessarily order dependent in their performance.

Unless stated otherwise as apparent from the following discussion, it will be appreciated that terms such as “segmenting,” “generating,” “registering,” “determining,” “aligning,” “positioning,” “processing,” “computing,” “selecting,” “estimating,” “detecting,” “tracking” or the like may refer to the actions and processes of a computer system, or similar electronic computing device, that manipulates and transforms data represented as physical (e.g., electronic) quantities within the computer system's registers and memories into other data similarly represented as physical quantities within the computer system memories or registers or other such information storage, transmission or display devices. Embodiments of the methods described herein may be implemented using computer software. If written in a programming language conforming to a recognized standard, sequences of instructions designed to implement the methods can be compiled for execution on a variety of hardware platforms and for interface to a variety of operating systems. In addition, implementations of the present framework are not described with reference to any particular programming language. It will be appreciated that a variety of programming languages may be used.

For brevity, an image, or a portion thereof (e.g., a region of interest (ROI) in the image) corresponding to an object (e.g., a tissue, an organ, a tumor, etc., of a subject (e.g., a patient, etc.)) may be referred to as an image, or a portion of thereof (e.g., an ROI) of or including the object, or the object itself. For instance, an ROI corresponding to the image of a lung or a heart may be described as that the ROI includes a lung or a heart. As another example, an image of or including a chest may be referred to a chest image, or simply a chest. For brevity, that a portion of an image corresponding to an object is processed (e.g., extracted, segmented) may be described as the object is processed. For instance, that a portion of an image corresponding to a lung is extracted from the rest of the image may be described as that the lung is extracted.

A framework for attenuation correction is presented herein. In accordance with one aspect, deep artificial neural networks (e.g., convolutional neural networks or CNNs) are trained for attenuation correction (AC) of emission image data (e.g., PET or SPECT). In some implementations, elastic registration is performed by the trained neural networks to match anatomic image data (e.g., CT image data) to NAC emission image data before performing attenuation correction (AC). In other implementations, synthetic (or pseudo) anatomic image data is generated by the trained neural networks based on the NAC emission image data to serve as the AC map. Attenuation correction may then be performed on the NAC emission image data using the synthetic anatomic image data.

Both approaches have the potential to improve current protocols using deep learning. They may be more robust, fully automated and obviate the need for CT scan following the stress scan. Advantageously, they can be used to reduce dose accumulation and/or total scan time, thereby reducing the associated risks. Additionally, AC quantification errors in clinical protocols may be reduced. Efficiency in the technologist workflow may be enhanced. These and other exemplary advantages and features will be described in more details in the following description.

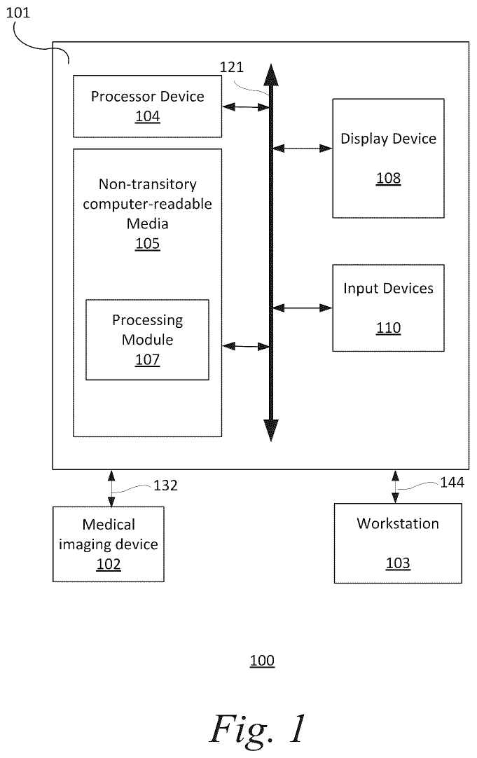

FIG. 1 is a block diagram illustrating an exemplary system 100. The system 100 includes a computer system 101 for implementing the framework as described herein. In some implementations, computer system 101 operates as a standalone device. In other implementations, computer system 101 may be connected (e.g., using a network) to other machines, such as medical imaging device 102 and workstation 103. In a networked deployment, computer system 101 may operate in the capacity of a server (e.g., in a server-client user network environment, a client user machine in server-client user network environment, or as a peer machine in a peer-to-peer (or distributed) network environment).

In one implementation, computer system 101 includes a processor device or central processing unit (CPU) 104 coupled to one or more non-transitory computer-readable media 105 (e.g., computer storage or memory device), display device 108 (e.g., monitor) and various input devices 110 (e.g., mouse, touchpad or keyboard) via an input-output interface 121. Computer system 101 may further include support circuits such as a cache, a power supply, clock circuits and a communications bus. Various other peripheral devices, such as additional data storage devices and printing devices, may also be connected to the computer system 101.

The present technology may be implemented in various forms of hardware, software, firmware, special purpose processors, or a combination thereof, either as part of the microinstruction code or as part of an application program or software product, or a combination thereof, which is executed via the operating system. In some implementations, the techniques described herein are implemented as computer-readable program code tangibly embodied in one or more non-transitory computer-readable media 105. In particular, the present techniques may be implemented by a processing module 107. Non-transitory computer-readable media 105 may include random access memory (RAM), read-only memory (ROM), magnetic floppy disk, flash memory, and other types of memories, or a combination thereof. The computer-readable program code is executed by processor device 104 to process data provided by, for example, medical imaging device 102. As such, the computer system 101 is a general-purpose computer system that becomes a specific-purpose computer system when executing the computer-readable program code. The computer-readable program code is not intended to be limited to any particular programming language and implementation thereof. It will be appreciated that a variety of programming languages and coding thereof may be used to implement the teachings of the disclosure contained herein. The same or different computer-readable media 105 may be used for storing a database, including, but not limited to, image datasets, a knowledge base, individual subject data, medical records, diagnostic reports (or documents) for subjects, or a combination thereof.

Medical imaging device 102 acquires image data 132. Such image data 132 may be processed by processing module 107. Medical imaging device 102 may be a radiology scanner (e.g., nuclear medicine scanner) and/or appropriate peripherals (e.g., keyboard and display device) for acquiring, collecting and/or storing such image data 132. Medical imaging device 102 may be a hybrid modality designed for acquiring image data using at least one anatomic imaging modality (e.g., CT, MR) and at least one molecular imaging modality (e.g., SPECT, PET). Anatomic imaging modality focuses on extracting structural information, while molecular imaging modality focuses on extracting functional information from molecules of interest. Medical imaging device 102 may be, for instance, a PET/CT, SPECT/CT or PET/MR scanner.

Workstation 103 may include a computer and appropriate peripherals, such as a keyboard and display device, and can be operated in conjunction with the entire system 100. For example, workstation 103 may communicate with medical imaging device 102 so that the medical image data 132 can be presented or displayed at the workstation 103. The workstation 103 may communicate directly with the computer system 101 to display processed data and/or output results 144. The workstation 103 may include a graphical user interface to receive user input via an input device (e.g., keyboard, mouse, touch screen, voice or video recognition interface, etc.) to manipulate visualization and/or processing of the data.

It is to be further understood that, because some of the constituent system components and method steps depicted in the accompanying figures can be implemented in software, the actual connections between the systems components (or the process steps) may differ depending upon the manner in which the present framework is programmed. Given the teachings provided herein, one of ordinary skill in the related art will be able to contemplate these and similar implementations or configurations of the present framework.

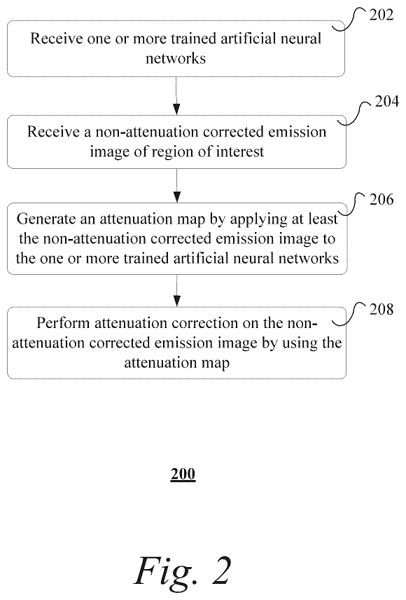

FIG. 2 shows an exemplary method 200 of attenuation correction. It should be understood that the steps of the method 200 may be performed in the order shown or a different order. Additional, different, or fewer steps may also be provided. Further, the method 200 may be implemented with the system 100 of FIG. 1, a different system, or a combination thereof.

At 202, processing module 107 receives one or more trained artificial neural networks (ANNs). The one or more artificial neural networks may be trained to improve and/or generate an attenuation map for attenuation correction of an emission image. The one or more artificial neural networks may include deep neural networks, such as convolutional neural networks (CNNs) or recurrent neural networks. The one or more artificial neural networks may include any architecture, such as the U-Net or the residual block network.

In some implementations, the one or more ANNs include at least one first neural network, at least one second neural network or a combination thereof. The at least one first neural network is trained using a training set of co-registered pairs of real anatomic images (e.g., CT, MR) and non-attenuation corrected emission images (e.g., PET, SPECT). For example, the at least one first neural network may be trained using co-registered pairs of CT and PET images. The registered real anatomic images in the training set provide ground truth for the training. The at least one first neural network is trained to perform an elastic registration of a real anatomic image to a non-attenuation corrected emission image to generate an attenuation map that is matched to the emission distribution.

The at least one second neural network may be trained using a training set of pairs of non-attenuation corrected emission image and linear attenuation coefficient map (μ-map or attenuation map) based on clinical subject data. The linear attenuation coefficient maps provide the ground truth for the training and may be calculated from the anatomic image data (e.g., CT data). The at least one second neural network is trained to generate a synthetic anatomic image based on the non-attenuation corrected emission image. The synthetic anatomic image is output as the attenuation map.

At 204, processing module 107 receives a non-attenuation corrected (NAC) emission image of a region of interest of a subject or patient. The region of interest may be any area identified for further study, such as the heart or lungs. The non-attenuation corrected emission image may be acquired by medical imaging device 102. Medical imaging device 102 may include a molecular imaging modality that directly acquires a non-attenuation corrected (NAC) emission image of the region of interest. To generate the emission image, the molecular imaging modality may detect emissions generated by a radioactive isotope injected into the subject's bloodstream. In some implementations, the emission image is a PET or SPECT image. Other types of molecular imaging modalities are also useful.

In some implementations, a real anatomic image of the region of interest is also received by processing module 107. The real anatomic image may be acquired by an anatomic imaging modality of medical imaging device 102. The real anatomic image may be, for example, a real CT or MR image. Other types of anatomic imaging modalities are also useful.

At 206, image processing module 117 generates an attenuation map by applying at least the non-attenuation corrected (NAC) emission image to the one or more trained artificial neural networks (ANNs). As discussed previously, the one or more trained ANNS may include at least one first neural network, at least one second neural network, or a combination thereof.

In some implementations, the at least one first neural network generates the attenuation map by performing an elastic registration of the real anatomic image to the NAC emission image to generate a registered anatomic image that is output as the attenuation map. Elastic registration is performed to spatially align the real anatomic image with the NAC emission image to generate the registered anatomic image. In some implementations, the at least one second neural network generates the attenuation map by constructing a synthetic (or pseudo) anatomic image of the region of interest based on the NAC emission mage that is output as the attenuation map.

In some implementations, a control unit preceding the first and second neural networks is provided to select either the first or second neural network for generating the attenuation map. The control unit may include a combinatorial digital circuit, a processor, a neural network, or any other type of computing circuit. When both a real anatomic image and the NAC emission image are provided as input to the control unit, the control unit may send or transmit the real anatomic image and NAC emission image to the at least one first neural network to generate the attenuation map. When only the NAC emission image (without the real anatomic image) is provided as input to the control unit, the control unit may send or transmit the input NAC emission image to the at least one second neural network to generate the attenuation map. In cases where the real anatomic image is provided but is not usable, the control unit may also send the input NAC emission image to the at least one second neural network for attenuation map generation.

At 208, image processing module 117 performs attenuation correction on the NAC emission image using the attenuation map generated by the one or more trained ANNs to generate an attenuation corrected emission image. More particularly, correction factors may be determined based on the attenuation map and used to correct the NAC emission image for attenuation, yielding the attenuation-corrected emission image. The attenuation corrected emission image may be displayed at, for example, workstation 103.

In some implementations, steps 204 through 208 may be repeated multiple times over time to generate a set of attenuation corrected emission images using a single real anatomic image. For example, the real anatomic image of the region of interest may be acquired once, while NAC emission images of the region of interest may be acquired at predetermined intervals (e.g., at different respiratory phases or cardiac positions).

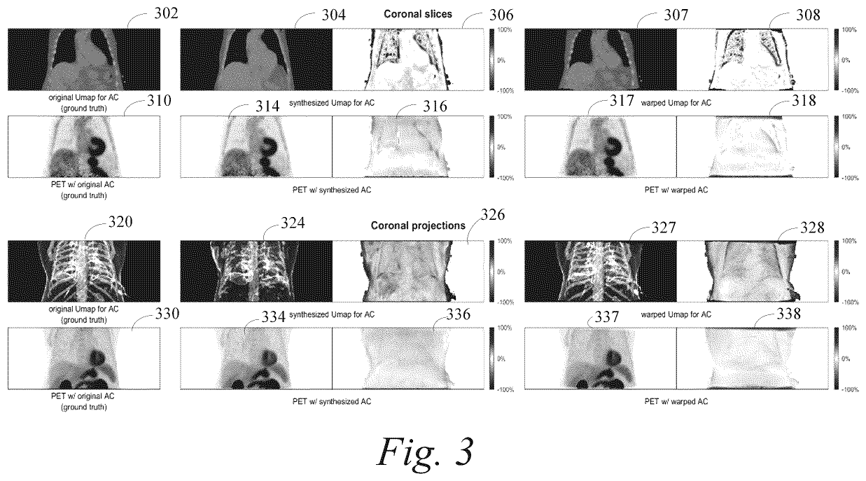

FIG. 3 illustrates an exemplary application of the present framework for a cardiac scan. The first and second rows of images show cardiac coronal slices of the test subject, while the third and fourth rows of images show cardiac coronal projections of the test subject. There is relatively good PET-CT image alignment in these images. The first row of images shows coronal slices of different μ-maps for attenuation correction. Synthetic CT image (attenuation or μ-map) 304 is generated by the one or more trained ANNs. Image 306 is the difference image obtained by subtracting the ground truth original μ-map 302 from the synthetic CT image 304, shown in terms of percentages of the original values in μ-map 302 obtained using a default AC method. Warped CT image (attenuation or μ-map) 307 is generated by the one or more trained ANNs. Image 308 is the difference image obtained by subtracting the ground truth original μ-map 302 from the warped CT image 307.

The second row of images shows the coronal slices of corresponding reconstructed PET images with attenuation correction. PET image 310 is the ground truth image that is attenuation corrected using the original μ-map 302. PET image 314 is attenuation corrected using the synthetic CT image 304. Image 316 is the difference image obtained by subtracting the ground truth image 310 from the PET image 314. PET image 317 is attenuation corrected using the warped CT image 307. Image 318 is the difference image obtained by subtracting the ground truth image 310 from the PET image 317.

The third row of images shows coronal projections of different μ-maps for attenuation correction. Synthetic CT image (attenuation or μ-map) 324 is generated by the one or more trained ANNs. Image 326 is the difference image obtained by subtracting the ground truth original μ-map 320 from the synthetic CT image 324, shown in terms of percentages of the original values in μ-map 320 obtained using a default AC method. Warped CT image (attenuation or μ-map) 327 is generated by the one or more trained ANNs. Image 328 is the difference image obtained by subtracting the ground truth original μ-map 320 from the warped CT image 327.

The fourth row of images shows the coronal projections of corresponding reconstructed PET images with attenuation correction. PET image 330 is the ground truth image that is attenuation corrected using the original μ-map 320. PET image 334 is attenuation corrected using the synthetic CT image 324. Image 336 is the difference image obtained by subtracting the ground truth image 330 from the PET image 334. PET image 337 is attenuation corrected using the warped CT image 327. Image 338 is the difference image obtained by subtracting the ground truth image 330 from the PET image 337.

Both approaches of generating the attenuation map provided reasonable attenuation correction for the heart, relative to the original CT image. In cases wherein the CT image is not aligned to the PET image, there is the possibility for the activity at certain locations in the myocardium to be under-corrected. This can create “false positive defects” in the reconstructed images. Both approaches for attenuation correction presented herein have the potential to avoid this. Furthermore, CT synthesis is well-positioned to address the CT truncation problem.

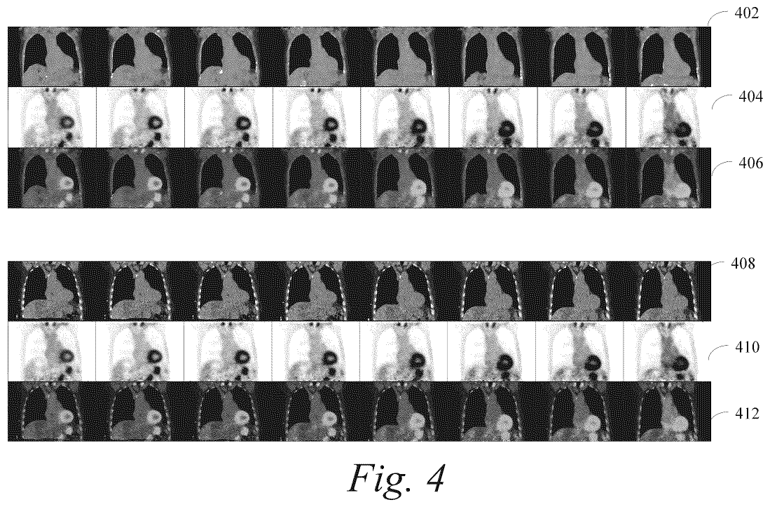

FIG. 4 shows an exemplary application of the present framework for a series of physiologically gated images. The first set of images (402, 404, 406) illustrates the use of synthetic CT images (attenuation or μ-maps) 402 for attenuation correction. The synthetic CT images 402 are generated by the one or more trained ANNs based on PET images acquired at different respiratory phases of a cardiac scan. Images 404 are PET images reconstructed with attenuation correction based on synthetic CT images 402. Images 406 show the PET images 404 overlaid on synthetic CT images 402.

The second set of images (408, 410, 412) illustrates the use of warped (or registered) CT images (attenuation or μ-maps) 408 for attenuation correction. The warped CT images 408 are generated by the one or more trained ANNs based on a single CT image and PET images acquired at different respiratory phases of a cardiac scan. Images 410 are PET images reconstructed with attenuation correction based on warped CT images 408. Images 412 show the PET images 410 overlaid on warped CT images 408.

While the present framework has been described in detail with reference to exemplary embodiments, those skilled in the art will appreciate that various modifications and substitutions can be made thereto without departing from the spirit and scope of the invention as set forth in the appended claims. For example, elements and/or features of different exemplary embodiments may be combined with each other and/or substituted for each other within the scope of this disclosure and appended claims.

What is claimed is:

1. An image processing system, comprising:
a non-transitory memory device for storing computer readable program code; and
a processor device in communication with the non-transitory memory device, the processor device being operative with the computer readable program code to perform steps including
(i) receiving one or more artificial neural networks,
(ii) receiving a non-attenuation corrected emission image of a region of interest,
(iii) generating an attenuation map by applying at least the non-attenuation corrected emission image to the one or more artificial neural networks, and
(iv) performing attenuation correction on the non-attenuation corrected emission image by using the attenuation map.

2. The image processing system of claim 1 wherein the one or more artificial neural networks comprise at least one first neural network, at least one second neural network or a combination thereof.

3. The image processing system of claim 2 wherein the processor device is operative with the computer readable program code to generate the attenuation map by applying a real anatomic image and the non-attenuation corrected emission image of the region of interest to the at least one first neural network.

4. The image processing system of claim 3 wherein the at least one first neural network performs an elastic registration of the real anatomic image to the non-attenuation corrected emission image to generate a registered anatomic image, wherein the registered anatomic image is output as the attenuation map.

5. The image processing system of claim 4 wherein the real anatomic image comprises a computed tomography (CT) or magnetic resonance (MR) image.

6. The image processing system of claim 2 wherein the at least one second neural network generates a synthetic anatomic image based on the non-attenuation corrected emission image, wherein the synthetic anatomic image is output as the attenuation map.

7. The image processing system of claim 1 wherein the non-attenuation corrected emission image comprises a positron-emission tomography (PET) or single-photon emission computerized tomography (SPECT) image.

8. The image processing system of claim 1 wherein the one or more artificial neural networks comprise one or more convolutional neural networks.

9. The image processing system of claim 1 wherein the processor device is operative with the computer readable program code to train the one or more artificial neural networks using a training set of co-registered pairs of real anatomic images and non-attenuation corrected emission images.

10. The image processing system of claim 1 wherein the processor device is operative with the computer readable program code to train the one or more artificial neural networks using a training set of pairs of non-attenuation corrected emission images and linear attenuation coefficient maps.

11. An image processing method, comprising:
(i) receiving one or more artificial neural networks;
(ii) receiving a non-attenuation corrected emission image of a region of interest;
(iii) generating an attenuation map by applying at least the non-attenuation corrected emission image to the one or more artificial neural networks; and
(iv) performing attenuation correction on the non-attenuation corrected emission image by using the attenuation map.

12. The image processing method of claim 11 further comprising applying a real anatomic image of the region of interest to the one or more artificial neural networks.

13. The image processing method of claim 12 wherein the one or more artificial neural networks perform an elastic registration of the real anatomic image to the non-attenuation corrected emission image to generate a registered anatomic image, wherein the registered anatomic image is output as the attenuation map.

14. The image processing method of claim 12 wherein the real anatomic image comprises a computed tomography (CT) or magnetic resonance (MR) image.

15. The image processing method of claim 11 wherein the one or more artificial neural networks generate a synthetic anatomic image based on the non-attenuation corrected emission image, wherein the synthetic anatomic image is output as the attenuation map.

16. The image processing method of claim 11 wherein the non-attenuation corrected emission image comprises a positron-emission tomography (PET) or single-photon emission computerized tomography (SPECT) image.

17. The image processing method of claim 11 further comprising training the one or more artificial neural networks using a training set of co-registered pairs of real anatomic images and non-attenuation corrected emission images.

18. The image processing method of claim 11 further comprising training the one or more artificial neural networks using a training set of pairs of non-attenuation corrected emission images and linear attenuation coefficient maps.

19. One or more non-transitory computer-readable media embodying instructions executable by a machine to perform operations for image processing comprising:
(i) receiving one or more artificial neural networks;
(ii) receiving a non-attenuation corrected emission image of a region of interest;
(iii) generating an attenuation map by applying at least the non-attenuation corrected emission image to the one or more artificial neural networks; and
(iv) performing attenuation correction on the non-attenuation corrected emission image by using the attenuation map.

20. The one or more non-transitory computer-readable media of claim 19 wherein the one or more artificial neural networks comprise a U-Net or residual block network.
